(19) United States
(12) Patent Application Publication
(10) Pub. No.: US 2017/0185759 A1
(43) Pub. Date: Jun. 29, 2017

(54) EMG-BASED LIVENESS DETECTION

(71) Applicants: Michael L. Schmidt, Beaverton, OR (US); Indira Negi, San Jose, CA (US); Alberto Vidal, San Jose, CA (US)

(72) Inventors: Michael L. Schmidt, Beaverton, OR (US); Indira Negi, San Jose, CA (US); Alberto Vidal, San Jose, CA (US)

(21) Appl. No.: 14/757,619

(22) Filed: Dec. 23, 2015

(51) Int. Cl. G06F 21/32; A61B 5/00; A61B 5/0492

(52) U.S. Cl. CPC G06F 21/32; A61B 5/0492; A61B 5/681; A61B 5/7203

(57) ABSTRACT

Various systems and methods for implementing EMG-based liveness detection are described herein. An EMG-based liveness detection system incorporated into a wearable device includes a plurality of electrodes to sense EMG signal data of a user; an amplifier to amplify the EMG signal data; a filter to remove signal noise from the EMG signal data; an analog-to-digital converter to analyze the EMG signal data and convert it to digital data; and a controller to: authenticate the user; perform an EMG-based liveness test of the user based on the digital data; and disable the wearable device when the EMG-based liveness test fails.

### TECHNICAL FIELD

Embodiments described herein generally relate to security interfaces and in particular, to EMG-based liveness detection.

### BACKGROUND

A spoof attack is a type of attack where the attacker presents an artificial authentication token in an attempt to circumvent a security system. In the case of a biometric authentication token, such as a fingerprint, a spoof attack may be executed using an artificial finger, a high-resolution image of a fingerprint, or even a sensor-level attack where the attacker feeds data to the sensor in an attempt to fool the sensor. To combat spoof attacks, one mechanism used is called liveness detection. In addition to verifying the authentication token, liveness detection also attempts to determine that the subject presenting the token is alive, distinct from an inanimate object, or dead person.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 is a schematic diagram illustrating an EMG sensor, according to an embodiment;

FIG. 2 is a schematic diagram of a wearable device, according to an embodiment;

FIG. 3 is a diagram illustrating electrode placement on the body and components of an EMG detector, according to an embodiment;

FIG. 4 is a pair of graphs illustrating test results, according to an embodiment;

FIG. 5 is a flowchart illustrating control and data flow, according to an embodiment;

FIG. 6 is a block diagram illustrating an EMG-based liveness detection system incorporated into a wearable device, according to an embodiment;

FIG. 7 is a flowchart illustrating a method 700 of implementing EMG-based liveness detection, according to an embodiment;

FIG. 8 is a block diagram illustrating an example machine upon which any one or more of the techniques (e.g., methodologies) discussed herein may perform, according to an example embodiment.

### DETAILED DESCRIPTION

In the following description, for purposes of explanation, numerous specific details are set forth in order to provide a thorough understanding of some example embodiments. It will be evident, however, to one skilled in the art that the present disclosure may be practiced without these specific details.

Systems and methods described herein implement EMG-based liveness detection. Liveness detection is important on a wearable device to determine whether the device is worn by a person or merely placed on an inanimate object. Liveness may be determined using a variety of mechanisms involving biometrics, such as optical heart rate monitoring or by analyzing an ECG (electrocardiogram) signal. However, optical heart rate monitoring for liveness detection is power intensive because it requires one or more LEDs to detect pulse. Any ECG-based liveness test requires the two electrodes on the device to be across the heart. In a wrist-based device being worn on one wrist, the user would have to touch it with her other hand, which is an inconvenient user experience. Embodiments described in this disclosure relate to the use of EMG (electromyography) on the wrist for liveness detection.

EMG is a technique for recording the electrical potential generated by muscle cells when these cells are neurologically activated. In a human body these action potentials are produced by the central nervous system and can only be present in a living person. If an external force is moving a part of the body, but the muscle is not activated by the brain and an EMG signal for the muscle to produce that motion will not be present. For example, if the wearer of a wearable EMG monitor lifts their finger, an EMG signal is measurable at the wrist, but if the same motion is caused by another force lifting the wearer's finger, no EMG signal is present.

Using EMG saves on compute requirements as well as power when compared to ECG or optical liveness detection mechanisms. EMG also saves on BOM (bill of materials) cost as well because the electrodes are just exposed metal parts in the enclosure of the wrist-based device, and do not require additional hardware like LEDs of as much amplification and filtering as ECG techniques. Further, EMG is a stronger signal than other electrode based biosignals like ECG, so it is easier to filter and separate from noise. This reduces the amount of signal processing required for liveness detection and makes EMG a promising candidate for on-device liveness detection. In addition, EMG techniques allow for faster processing and lower MIPS (million instructions per second) usage.

EMG is better than optical methods in terms of the power consumed by hardware for signal acquisition. LEDs are very power hungry, and a single LED for liveness uses ˜300 mA for each sampling, and ˜30-50 Hz is typical sampling frequency. By comparison, an EMG signal may be sampled at 700 Hz and uses under 5 mA per sample. This results in a significant improvement over EMG power consumption. The EMG signal typically resides between 10-350 Hz. As such, the signal may be filtered below 10 Hz in order to remove movement artifacts from the EMG signal.

Biometric liveness detectors are generally better than other types of detectors, such as proximity sensors. A proximity sensor may be used for liveness detection, but cannot distinguish between being proximate to human skin or an inanimate object.

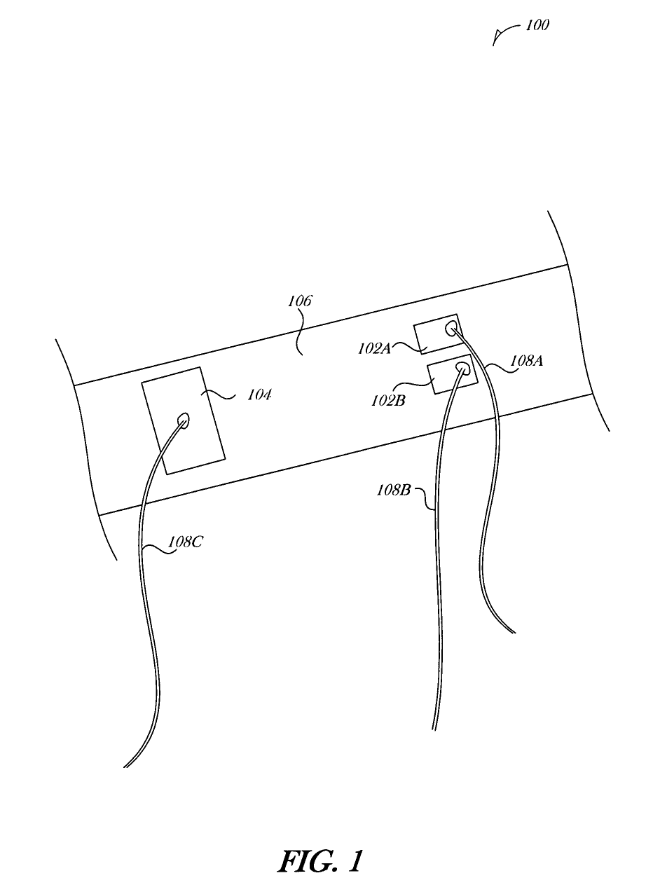

FIG. 1 is a schematic diagram illustrating an EMG sensor 100, according to an embodiment. The EMG sensor includes two EMG signal electrodes 102A, 102B, and a reference electrode 104 attached to a wrist strap 106. The electrodes 102A, 102B, 104 may be constructed from conductive tape and adhered to a strip of urethane foam. Solder joints connecting wires 108A, 108B, 108C soldered to the electrodes 102A, 102B, 104, respectively, may be insulated with Kapton® tape to ensure that signals are acquired from the electrodes 102A, 102B, 104, and not from the wires 108A, 108B, 108C. The strap 106 may be integrated into a wrist-worn device, such as a smartwatch.

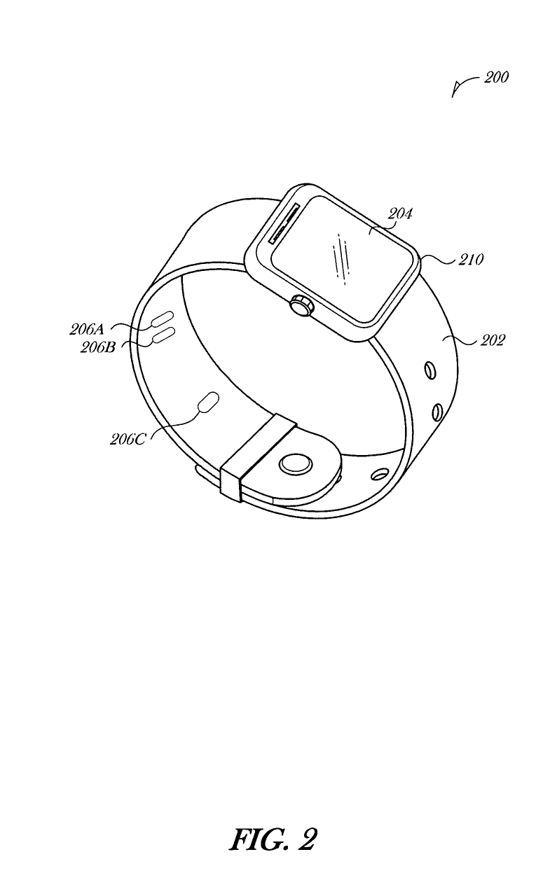

FIG. 2 is a schematic diagram of a wearable device 200, according to an embodiment. The wearable device 200 includes a wrist band 202, a display 204, and electrodes 206A, 206B, 206C. Electrodes 206A, 206B may be used as EMG signal electrodes and electrode 206C may be used as a reference electrode. The display 204 may be used to present various information, such as the time, email notifications, text, etc. to the user. The electrodes 206A, 206B, 206C may be positioned to acquire EMG signals from the user's wrist while the wearable device 200 is worn.

A user may be prompted via the display 204 to adjust the wearable device 200 during initialization, to properly align the electrodes 206. The prompt may be provided after the user has already been authenticated, such as with a username/password combination, PIN, or other security feature. During initialization, the electrodes 206 may capture EMG signals to ensure liveness.

If the user decides to wear the wearable device 200 in a different place, such as the alternative wrist, the user may manually initiate the initialization process to adjust the positon of the wearable device 200. The user may execute such functions from a secured component of the wearable device 200, such as a configuration user interface provided to the user after being logged in with a username/password combination, biometric authentication, a PIN, or the like.

While three electrodes 206 are illustrated in FIG. 2, it is understood that many more electrodes may be integrated into the wrist band 202 or back of housing 210. A larger number of electrodes (e.g., 10, 15, 20, etc.) may be used and electrically selected to operate as EMG signal electrodes or reference electrodes. In this manner, the user is able to wear the wearable device 200 in any position (e.g., upside down on their wrist with the watch face facing down), and electrodes may be electrically selected by the wearable device 200 to perform liveness detection.

While FIG. 2 illustrates the wearable device 102 as a wrist-worn device, it is understood that the wearable device 102 may be any type of wearable able to sense EMG signals, such as glasses, mask, shirt, socks, pants, gloves, or any e-textile that contacts the skin. For example, in a glasses-based device, the electrodes may be incorporated into a temple of the wearable device, such that movement of muscles around the side of the head or the ear may be detected.

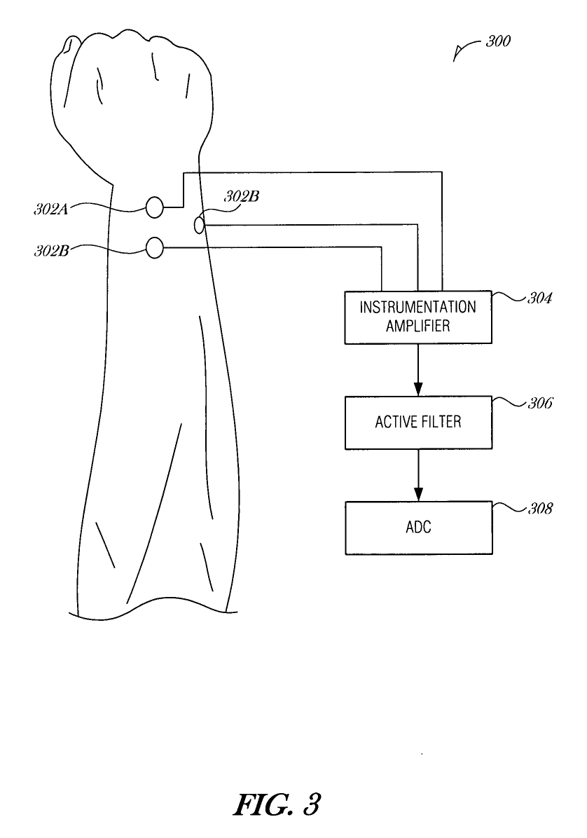

FIG. 3 is a diagram illustrating electrode placement on the body and components of an EMG detector 300, according to an embodiment. Signal electrodes 302A, 302B and reference electrode 302C may be fed into a high input impedance instrumentation amplifier 304. A bipolar electrode configuration is used for its improved noise rejection over a unipolar electrode, although a unipolar electrode configuration may be used in some embodiments. In addition to the bipolar electrodes 302A, 302B, an additional reference electrode 302C is added to further improve the noise rejection of the system. The amplified EMG signal is then further amplified and filtered to 10-350 Hz with an active Butterworth filter 306, before being sampled by an analog-to-digital converter (ADC) 308. The output from the ADC 308 may be analyzed to determine whether a signal having more than a threshold amount of EMG amplitude is sensed.

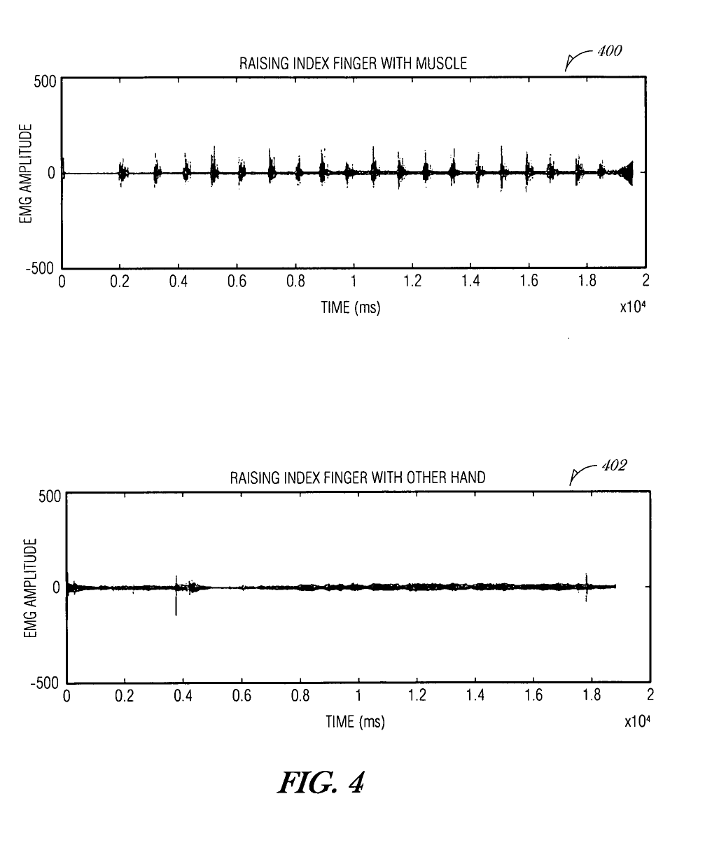

FIG. 4 is a pair of graphs 400, 402 illustrating test results, according to an embodiment. Graph 400 shows results of when an index finger is flexed upwards by the subject, activating the various posterior muscles in the forearm. Graph 402 shows results of movement of the index finger, the movement caused by an external force (e.g., raised by another person). As may be observed, there is distinct signals found in the graph 400 where the person raised his own finger in contrast to the second graph 402, where the signal is hardly distinguishable from noise.

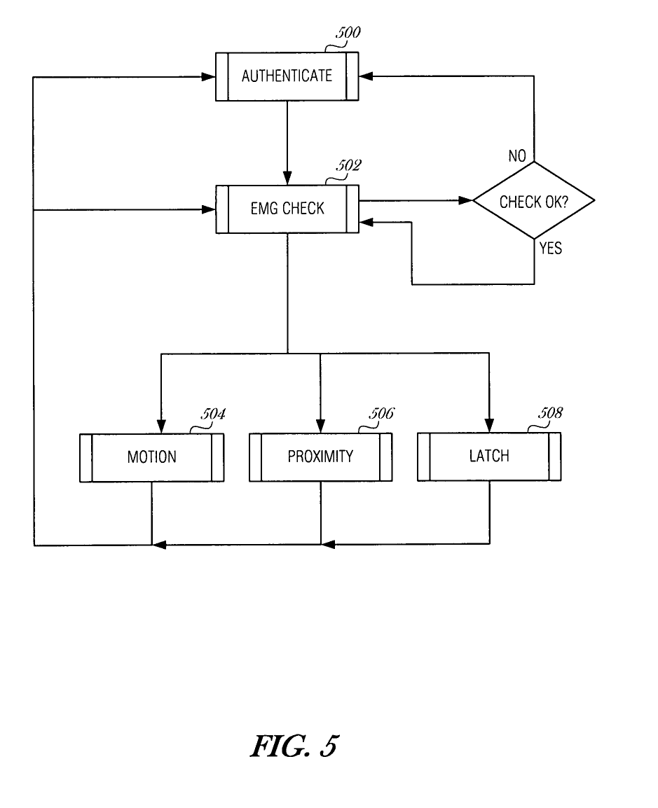

FIG. 5 is a flowchart illustrating control and data flow, according to an embodiment. A user may authenticate with a wearable device (operation 500). The authentication may be by any mechanism, such as a username and password combination, a PIN, a biometric scan (e.g., fingerprint scan), by use with another device (e.g., a secure fob and proximity security), or combination thereof.

At regular intervals, an EMG check is performed (operation 502). The EMG check may be performed when the user initially places the wearable device on their body. For example, the EMG check may be initiated by a proximity check, such that when a proximity sensor detects that the wearable device is on or near a surface, the EMG check is initiated. As another example, the EMG check may be performed when the wearable device detect movement or motion (e.g., when the device is picked up or when the user moves). As another example, the wearable device may detect when a clasp or buckle on a wristband is latched and unlatched. When the clasp is initially latched, an EMG check may be performed to ensure that the wearable device is affixed to a live person.

When an event occurs, the flow returns to the authentication operation 500 or the EMG check 502, in various embodiments. The event may be when the person takes off the wearable device, such as may be detected using motion based detection (operation 504) or by proximity detection (operation 506). In a related example, a sensor may be used to detect that a clasp, latch, buckle, or other latching mechanism has been released by the user (operation 508).

When the user un-equips the wearable device, the user may have to re-authenticate. Depending on the policy being enforced, the user may not have to re-authenticate, but instead an EMG check (operation 502) may be performed after the user re-equips the wearable device.

If the EMG check fails, then the wearable device may lock, power off, or otherwise disable itself based on policies in force. In some embodiments, if the EMG check fails, the wearable device may lock so that the user has to re-authenticate.

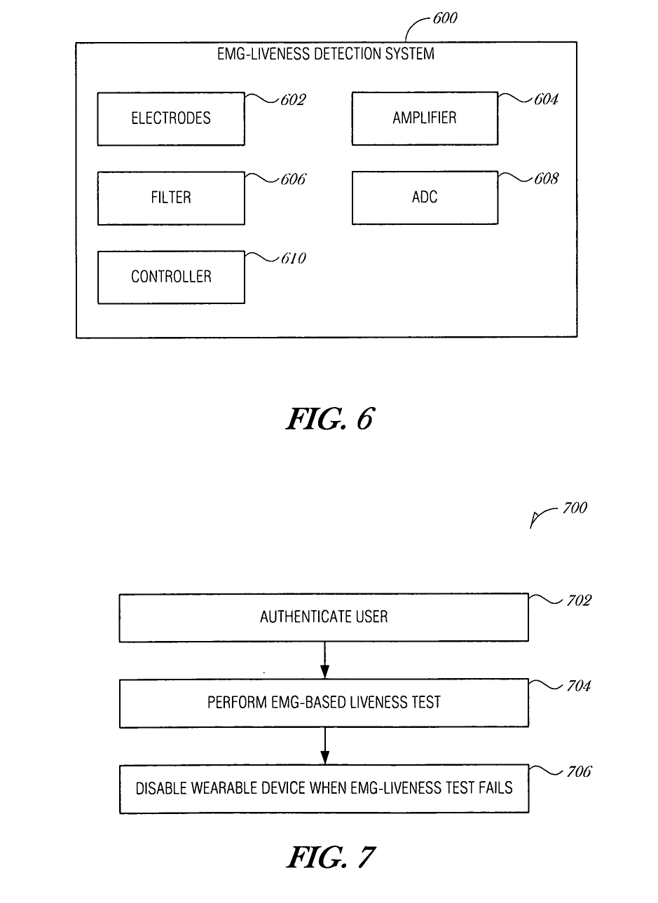

FIG. 6 is a block diagram illustrating an EMG-based liveness detection system 600 incorporated into a wearable device, according to an embodiment. The system 600 may include a plurality of electrodes 602 to sense EMG signal data of a user; an amplifier 604 to amplify the EMG signal data; a filter 606 to remove signal noise from the EMG signal data; an analog-to-digital converter 608 to analyze the EMG signal data and convert it to digital data; and a controller 610. In an embodiment, the wearable device comprises a smartwatch, although it is understood that any wearable device may be used, including but not limited to conductive fabric clothes, smart glasses, smart shoes, smart bracelet, smart rings, etc.

The controller 610 may be configured to authenticate the user. In an embodiment, to authenticate the user, the controller 610 is to authenticate the user with a username and password authentication mechanism. In another embodiment, to authenticate the user, the controller 610 is to authenticate the user with a biometric authentication mechanism.

The controller 610 may also be configured to perform an EMG-based liveness test of the user based on the digital data. In an embodiment, to perform the EMG-based liveness test, the controller 610 is to sense an EMG signal from the user and determine whether the EMG signal indicates that the user has activated a muscle near the wearable device. In a further embodiment, sensing the EMG signal from the user comprises using a bipolar electrode configuration. A unipolar electrode configuration may also be used. In an embodiment, performing the EMG-based liveness test is performed periodically. For example, the EMG-based liveness test may be performed every 30 seconds, or at some other interval. The interval may be configurable by the user.

The controller 610 may also be configured to disable the wearable device when the EMG-based liveness test fails. In an embodiment, to disable the wearable device, the controller 610 is to lock the wearable device. In another embodiment, to disable the wearable device, the controller 610 is to power down the wearable device.

In an embodiment, the controller 610 is to disable the wearable device upon the occurrence of an event and re-authenticate the user at the wearable device. In a related embodiment, the event comprises motion that indicates the user removed the wearable device from their body. In a related embodiment, the event comprises proximity sensor data that indicates the user removed the wearable device from their body. In a related embodiment, the event comprises data that indicates the user removed unlatched the wearable device. Other events may be used to reset the authentication or have the user re-establish the liveness state, such as a timeout (e.g., screen lock after five minutes of idle activity), a remote lock (e.g., locking the wearable device from another computer), or a software control on the wearable device (e.g., software may detect unusual activity and perform remedial actions to secure the wearable device).

FIG. 7 is a flowchart illustrating a method 700 of implementing EMG-based liveness detection, according to an embodiment. At block 702, a user is authenticated at a wearable device. In an embodiment, authenticating the user comprises authenticating the user with a username and password authentication mechanism. In a related embodiment, authenticating the user comprises authenticating the user with a biometric authentication mechanism.

The wearable device may be any type of wearable computing equipment including a smartwatch, smart ring, smart glasses, e-textile, or the like. In an embodiment, the wearable device comprises a smartwatch.

At block 704, an EMG-based liveness test of the user is performed. In an embodiment, performing the EMG-based liveness test comprises sensing an EMG signal from the user and determining whether the EMG signal indicates that the user has activated a muscle near the wearable device. In a further embodiment, sensing the EMG signal from the user comprises using a bipolar electrode configuration. A unipolar electrode configuration may alternatively be used.

In an embodiment, performing the EMG-based liveness test is performed periodically. For example, the EMG-based liveness test may be performed every 30 seconds, or at some other interval. The interval may be configurable by the user.

At block 706, the wearable device is disabled when the EMG-based liveness test fails. In an embodiment, disabling the wearable device comprises locking the wearable device. In a related embodiment, disabling the wearable device comprises powering down the wearable device.

In an embodiment, the method 700 includes disabling the wearable device upon the occurrence of an event and re-authenticating the user at the wearable device. In a related embodiment, the event comprises motion that indicates the user removed the wearable device from their body. In a related embodiment, the event comprises proximity sensor data that indicates the user removed the wearable device from their body. In a related embodiment, the event comprises data that indicates the user removed unlatched the wearable device.

Embodiments may be implemented in one or a combination of hardware, firmware, and software. Embodiments may also be implemented as instructions stored on a machine-readable storage device, which may be read and executed by at least one processor to perform the operations described herein. A machine-readable storage device may include any non-transitory mechanism for storing information in a form readable by a machine (e.g., a computer). For example, a machine-readable storage device may include read-only memory (ROM), random-access memory (RAM), magnetic disk storage media, optical storage media, flash-memory devices, and other storage devices and media.

A processor subsystem may be used to execute the instruction on the machine-readable medium. The processor subsystem may include one or more processors, each with one or more cores. Additionally, the processor subsystem may be disposed on one or more physical devices. The processor subsystem may include one or more specialized processors, such as a graphics processing unit (GPU), a digital signal processor (DSP), a field programmable gate array (FPGA), or a fixed function processor.

Examples, as described herein, may include, or may operate on, logic or a number of components, modules, or mechanisms. Modules may be hardware, software, or firmware communicatively coupled to one or more processors in order to carry out the operations described herein. Modules may be hardware modules, and as such modules may be considered tangible entities capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine-readable medium. In an example, the software, when executed by the underlying hardware of the module, causes the hardware to perform the specified operations. Accordingly, the term hardware module is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software; the general-purpose hardware processor may be configured as respective different modules at different times. Software may accordingly configure a hardware processor, for example, to constitute a particular module at one instance of time and to constitute a different module at a different instance of time. Modules may also be software or firmware modules, which operate to perform the methodologies described herein.

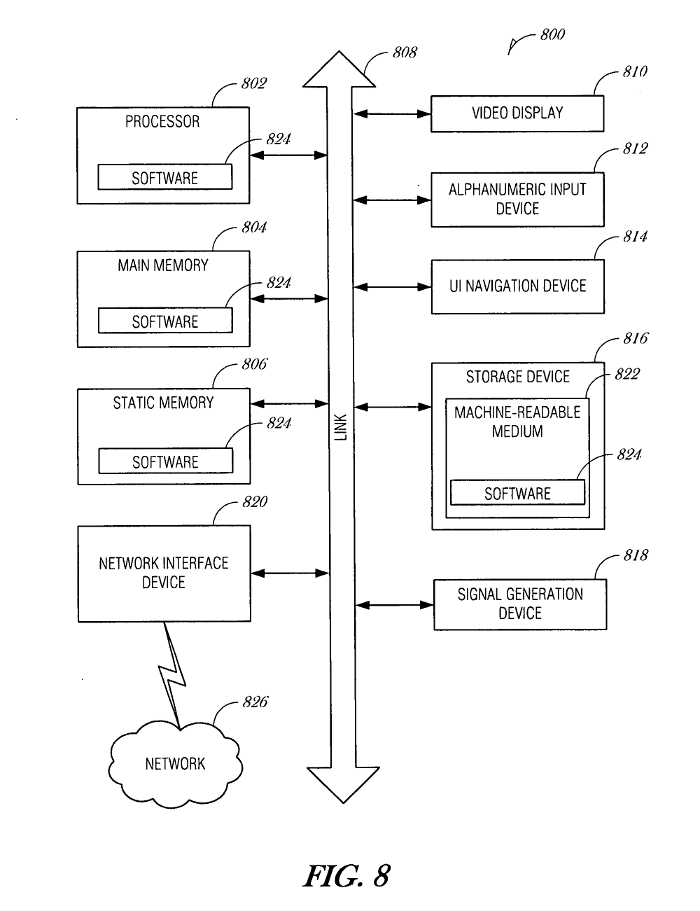

FIG. 8 is a block diagram illustrating a machine in the example form of a computer system 800, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be an onboard vehicle system, wearable device, personal computer (PC), a tablet PC, a hybrid tablet, a personal digital assistant (PDA), a mobile telephone, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 800 includes at least one processor 802 (e.g., a central processing unit (CPU), a graphics processing unit (GPU) or both, processor cores, compute nodes, etc.), a main memory 804 and a static memory 806, which communicate with each other via a link 808 (e.g., bus). The computer system 800 may further include a video display unit 810, an alphanumeric input device 812 (e.g., a keyboard), and a user interface (UI) navigation device 814 (e.g., a mouse). In one embodiment, the video display unit 810, input device 812 and UI navigation device 814 are incorporated into a touch screen display. The computer system 800 may additionally include a storage device 816 (e.g., a drive unit), a signal generation device 818 (e.g., a speaker), a network interface device 820, and one or more sensors (not shown), such as a global positioning system (GPS) sensor, compass, accelerometer, gyrometer, magnetometer, or other sensor.

The storage device 816 includes a machine-readable medium 822 on which is stored one or more sets of data structures and instructions 824 (e.g., software) embodying or utilized by any one or more of the methodologies or functions described herein. The instructions 824 may also reside, completely or at least partially, within the main memory 804, static memory 806, and/or within the processor 802 during execution thereof by the computer system 800, with the main memory 804, static memory 806, and the processor 802 also constituting machine-readable media.

While the machine-readable medium 822 is illustrated in an example embodiment to be a single medium, the term “machine-readable medium” may include a single medium or multiple media (e.g., a centralized or distributed database, and/or associated caches and servers) that store the one or more instructions 824. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Bluetooth, Wi-Fi, 3G, and 4G LTE/LTE-A or WiMAX networks). The term “transmission medium” shall be taken to include any intangible medium that is capable of storing, encoding, or carrying instructions for execution by the machine, and includes digital or analog communications signals or other intangible medium to facilitate communication of such software.

### ADDITIONAL NOTES & EXAMPLES

Example 1 includes subject matter (such as a device, apparatus, or machine) for implementing EMG-based liveness detection comprising: a plurality of electrodes to sense EMG signal data of a user; an amplifier to amplify the EMG signal data; a filter to remove signal noise from the EMG signal data; an analog-to-digital converter to analyze the EMG signal data and convert it to digital data; and a controller to: authenticate the user; perform an EMG-based liveness test of the user based on the digital data; and disable the wearable device when the EMG-based liveness test fails.

In Example 2, the subject matter of Example 1 may include, wherein to authenticate the user, the controller is to authenticate the user with a username and password authentication mechanism.

In Example 3, the subject matter of any one of Examples 1 to 2 may include, wherein to authenticate the user, the controller is to authenticate the user with a biometric authentication mechanism.

In Example 4, the subject matter of any one of Examples 1 to 3 may include, wherein the wearable device comprises a smartwatch.

In Example 5, the subject matter of any one of Examples 1 to 4 may include, wherein to perform the EMG-based liveness test, the controller is to: sense an EMG signal from the user; and determine whether the EMG signal indicates that the user has activated a muscle near the wearable device.

In Example 6, the subject matter of any one of Examples 1 to 5 may include, wherein sensing the EMG signal from the user comprises using a bipolar electrode configuration.

In Example 7, the subject matter of any one of Examples 1 to 6 may include, wherein performing the EMG-based liveness test is performed periodically.

In Example 8, the subject matter of any one of Examples 1 to 7 may include, wherein to disable the wearable device, the controller is to lock the wearable device.

In Example 9, the subject matter of any one of Examples 1 to 8 may include, wherein to disable the wearable device, the controller is to power down the wearable device.

In Example 10, the subject matter of any one of Examples 1 to 9 may include, wherein the controller is to: disable the wearable device upon the occurrence of an event; and re-authenticate the user at the wearable device.

In Example 11, the subject matter of any one of Examples 1 to 10 may include, wherein the event comprises motion that indicates the user removed the wearable device from their body.

In Example 12, the subject matter of any one of Examples 1 to 11 may include, wherein the event comprises proximity sensor data that indicates the user removed the wearable device from their body.

In Example 13, the subject matter of any one of Examples 1 to 12 may include, wherein the event comprises data that indicates the user removed unlatched the wearable device.

Example 14 includes subject matter (such as a method, means for performing acts, machine readable medium including instructions that when performed by a machine cause the machine to performs acts, or an apparatus to perform) for implementing EMG-based liveness detection comprising: authenticating a user at a wearable device; performing an EMG-based liveness test of the user; and disabling the wearable device when the EMG-based liveness test fails.

In Example 15, the subject matter of Example 14 may include, wherein authenticating the user comprises authenticating the user with a username and password authentication mechanism.

In Example 16, the subject matter of any one of Examples 14 to 15 may include, wherein authenticating the user comprises authenticating the user with a biometric authentication mechanism.

In Example 17, the subject matter of any one of Examples 14 to 16 may include, wherein the wearable device comprises a smartwatch.

In Example 18, the subject matter of any one of Examples 14 to 17 may include, wherein performing the EMG-based liveness test comprises: sensing an EMG signal from the user; and determining whether the EMG signal indicates that the user has activated a muscle near the wearable device.

In Example 19, the subject matter of any one of Examples 14 to 18 may include, wherein sensing the EMG signal from the user comprises using a bipolar electrode configuration.

In Example 20, the subject matter of any one of Examples 14 to 19 may include, wherein performing the EMG-based liveness test is performed periodically.

In Example 21, the subject matter of any one of Examples 14 to 20 may include, wherein disabling the wearable device comprises locking the wearable device.

In Example 22, the subject matter of any one of Examples 14 to 21 may include, wherein disabling the wearable device comprises powering down the wearable device.

In Example 23, the subject matter of any one of Examples 14 to 22 may include, disabling the wearable device upon the occurrence of an event; and re-authenticating the user at the wearable device.

In Example 24, the subject matter of any one of Examples 14 to 23 may include, wherein the event comprises motion that indicates the user removed the wearable device from their body.

In Example 25, the subject matter of any one of Examples 14 to 24 may include, wherein the event comprises proximity sensor data that indicates the user removed the wearable device from their body.

In Example 26, the subject matter of any one of Examples 14 to 25 may include, wherein the event comprises data that indicates the user removed unlatched the wearable device.

Example 27 includes at least one machine-readable medium including instructions, which when executed by a machine, cause the machine to perform operations of any of the Examples 14-26.

Example 28 includes an apparatus comprising means for performing any of the Examples 14-26.

Example 29 includes subject matter (such as a device, apparatus, or machine) for implementing EMG-based liveness detection comprising: means for authenticating a user at a wearable device; means for performing an EMG-based liveness test of the user; and means for disabling the wearable device when the EMG-based liveness test fails.

In Example 30, the subject matter of Example 29 may include, wherein the means for authenticating the user comprises means for authenticating the user with a username and password authentication mechanism.

In Example 31, the subject matter of any one of Examples 29 to 30 may include, wherein the means for authenticating the user comprises means for authenticating the user with a biometric authentication mechanism.

In Example 32, the subject matter of any one of Examples 29 to 31 may include, wherein the wearable device comprises a smartwatch.

In Example 33, the subject matter of any one of Examples 29 to 32 may include, wherein the means for performing the EMG-based liveness test comprises: means for sensing an EMG signal from the user; and means for determining whether the EMG signal indicates that the user has activated a muscle near the wearable device.

In Example 34, the subject matter of any one of Examples 29 to 33 may include, wherein the means for sensing the EMG signal from the user comprises means for using a bipolar electrode configuration.

In Example 35, the subject matter of any one of Examples 29 to 34 may include, wherein performing the EMG-based liveness test is performed periodically.

In Example 36, the subject matter of any one of Examples 29 to 35 may include, wherein the means for disabling the wearable device comprises means for locking the wearable device.

In Example 37, the subject matter of any one of Examples 29 to 36 may include, wherein the means for disabling the wearable device comprises means for powering down the wearable device.

In Example 38, the subject matter of any one of Examples 29 to 37 may include, means for disabling the wearable device upon the occurrence of an event; and means for re-authenticating the user at the wearable device.

In Example 39, the subject matter of any one of Examples 29 to 38 may include, wherein the event comprises motion that indicates the user removed the wearable device from their body.

In Example 40, the subject matter of any one of Examples 29 to 39 may include, wherein the event comprises proximity sensor data that indicates the user removed the wearable device from their body.

In Example 41, the subject matter of any one of Examples 29 to 40 may include, wherein the event comprises data that indicates the user removed unlatched the wearable device.

Example 42 includes subject matter (such as a device, apparatus, or machine) for implementing EMG-based liveness detection comprising: a processor subsystem; and a memory including instructions, which when executed by the processor subsystem, cause the processor subsystem to: authenticate a user at a wearable device; perform an EMG-based liveness test of the user; and disable the wearable device when the EMG-based liveness test fails.

In Example 43, the subject matter of Example 42 may include, wherein the instructions to authenticate the user comprise instructions to authenticate the user with a username and password authentication mechanism.

In Example 44, the subject matter of any one of Examples 42 to 43 may include, wherein the instructions to authenticate the user comprise instructions to authenticate the user with a biometric authentication mechanism.

In Example 45, the subject matter of any one of Examples 42 to 44 may include, wherein the wearable device comprises a smartwatch.

In Example 46, the subject matter of any one of Examples 42 to 45 may include, wherein the instructions to perform the EMG-based liveness test comprise instructions to: sense an EMG signal from the user; and determine whether the EMG signal indicates that the user has activated a muscle near the wearable device.

In Example 47, the subject matter of any one of Examples 42 to 46 may include, wherein the instructions to sense the EMG signal from the user comprise instructions to use a bipolar electrode configuration.

In Example 48, the subject matter of any one of Examples 42 to 47 may include, wherein the instructions to perform the EMG-based liveness test is performed periodically.

In Example 49, the subject matter of any one of Examples 42 to 48 may include, wherein the instructions to disable the wearable device comprise instructions to lock the wearable device.

In Example 50, the subject matter of any one of Examples 42 to 49 may include, wherein the instructions to disable the wearable device comprise instructions to powering down the wearable device.

In Example 51, the subject matter of any one of Examples 42 to 50 may include, instructions to: disable the wearable device upon the occurrence of an event; and re-authenticate the user at the wearable device.

In Example 52, the subject matter of any one of Examples 42 to 51 may include, wherein the event comprises motion that indicates the user removed the wearable device from their body.

In Example 53, the subject matter of any one of Examples 42 to 52 may include, wherein the event comprises proximity sensor data that indicates the user removed the wearable device from their body.

In Example 54, the subject matter of any one of Examples 42 to 53 may include, wherein the event comprises data that indicates the user removed unlatched the wearable device.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, also contemplated are examples that include the elements shown or described. Moreover, also contemplated are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to suggest a numerical order for their objects.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

What is claimed is:

1. An EMG-based liveness detection system incorporated into a wearable device, the system comprising:
a plurality of electrodes to sense EMG signal data of a user;
an amplifier to amplify the EMG signal data;
a filter to remove signal noise from the EMG signal data;
an analog-to-digital converter to analyze the EMG signal data and convert it to digital data; and
a controller to:
authenticate the user;
periodically perform an EMG-based liveness test of the user based on the digital data after the user has been authenticated;
disable the wearable device upon the occurrence of an event; and
re-authenticate the user at the wearable device.

2. The system of claim 1, wherein to authenticate the user, the controller is to authenticate the user with a username and password authentication mechanism.

3. The system of claim 1, wherein to authenticate the user, the controller is to authenticate the user with a biometric authentication mechanism.

4. The system of claim 1, wherein the wearable device comprises a smartwatch.

5. The system of claim 1, wherein to perform the EMG-based liveness test, the controller is to:
sense an EMG signal from the user; and
determine whether the EMG signal indicates that the user has activated a muscle near the wearable device.

6. The system of claim 5, wherein sensing the EMG signal from the user comprises using a bipolar electrode configuration.

7. The system of claim 1, wherein performing the EMG-based liveness test is performed periodically.

8. The system of claim 1, wherein o disable the wearable device, the controller is to lock the wearable device.

9. The system of claim 1, wherein to disable the wearable device, the controller is to power down the wearable device.

10. (canceled)

11. The system of claim 1, wherein the event comprises motion that indicates the user removed the wearable device from their body.

12. The system of claim 1, wherein the event comprises proximity sensor data that indicates the user removed the wearable device from their body.

13. The system of claim 1, wherein the event comprises data that indicates the user removed unlatched the wearable device.

14. A method of implementing EMG-based liveness detection, the method comprising:
authenticating, at a wearable device, a user of the wearable device;
periodically performing an EMG-based liveness test of the user after the user has been authenticated;
disabling the wearable device upon the occurrence of an event; and
re-authenticating the user at the wearable device.

15. The method of claim 14, wherein authenticating the user comprises authenticating the user with a username and password authentication mechanism.

16. The method of claim 14, wherein authenticating the user comprises authenticating the user with a biometric authentication mechanism.

17. The method of claim 14, wherein the wearable device comprises a smartwatch.

18. The method of claim 14, wherein performing the EMG-based liveness test comprises:
sensing an EMG signal from the user; and
determining whether the EMG signal indicates that the user has activated a muscle near the wearable device.

19. The method of claim 18, wherein sensing the EMG signal from the user comprises using a bipolar electrode configuration.

20. (canceled)

21. At least one non-transitory machine-readable medium including instructions, which when executed by a machine, cause the machine to
authenticate a user at a wearable device;
periodically perform an EMG-based liveness test of the user after the user has been authenticated;
disable the wearable device upon the occurrence of an event; and
re-authenticate the user at the wearable device.

22. The at least one machine-readable medium of claim 21, wherein the instructions to perform the EMG-based liveness test comprise instructions to:
sense an EMG signal from the user; and
determine whether the EMG signal indicates that the user has activated a muscle near the wearable device.

23-24. (canceled)

25. The at least one machine-readable medium of claim 21, wherein the event comprises motion that indicates the user removed the wearable device from their body.
